SEQUENCE LISTING (91 sequences)

SEQ ID NO: 1 - DNA, 912 bp, Sclerotinia homoeocarpa
  source 1..912: mol_type = genomic DNA
tagatctacacatggttcttacattatatttaggtcacttgatctacaagtgcggtggaattgacaagcgtactattgaaaagttcgagacggtatgacttctccacctttctcttgctatcttttcccgtccttctcatcgagatcagtgtctgcgatcttggtgctgatggatttatcgggttgcgttttctctcatgcgcggagcatacatccgaattctcaaccctttgaacattaccacattgcctttccagaatccctttgctaacccgttaataggaagccaaggagatgggaaagggttccttcaagtacgcatgggttttggacaagttgaaggctgagcgtgagcgtggtatcaccatcgacattgccctctggaagttcgagacacctaagtacaatgttactgtcattggtatgtgtacgaattctttatgccaactgaagtatattaacccattcgcagatgcccccggtcatcgtgatttcatcaagaacatgatcactggtacctcccaagctgattgtgccattcttatcatcgctgccggtgttggtgagttcgaggctggtatctccaaggatggtcagacccgtgagcacgctcttcttgcgtacactcttggtgttaagcaacttatcgttgccatcaacaagatggacaccaccaagtggtccaaggatcgtttcgaggaaatcatcaaggagacaaccaacttcatcaagaaggttggctacaacgccaagactgttcccttcgtgccgatctctggattcgagggtgataacatgattgagccctcaactaactgcccatggtacaagggctgggagagagagtccaaggagtctggcaaacacaccggcaagacccttcttgaggccatcgacagcatggacctgcct

SEQ ID NO: 2 - DNA, 629 bp, Rhizoctonia solani
  misc_feature 1..629: note = Rhizoctonia solani AG2-2IIIB
  source 1..629: mol_type = genomic DNA
tgtagctggctccattagtttggagcatgtgcacaccttttgctctttttttaatccacacacacctgtgaacctgtgaggcagagacatggatgggagaacttttatttactttaaaatgaatgattgggacccctacccccccccccctctgtctactcaactctaatataaacccaatttattttaaaatgaatgtaatggatgtaacgcatctaatactaagtttcaacaacggatctcttggctctcgcatcgatgaagaacgcagcgaaatgcgataagtaatgtgaattgcagaattcagtgaatcatcgaatctttgaacgcaccttgcgctccttggtattccttggagcatgcctgtttgagtatcatgaaatcttcaaagtaaacctttttgttaactcaatttggtttcactttggtattggaggttcttgcagcttcacacgctgctcctctttgttcattagctggatctcagtgttatgcttggttcctctcggcgtgataaattatctatcgctgaggactcccgataaaaaggttggccaaggtaaatgcagatgaaccgcttctaatagtccattgacttggacaataaaataattattattttacgatct

SEQ ID NO: 3 - DNA, 613 bp, Microdochium nivale
  misc_feature 1..613: note = Microdochium nivale var. nivale
  source 1..613: mol_type = genomic DNA
ggtaaccaaatcggtgctgctttctggtgcgtacacctcgactcgaagacgaccacgaccttcgcgacgaaaatgaactcggcagccaaaaaccgtgccgtcgagaatctttagtcgcagaggaatctaacataagggtggagaccggcaaggctaacactatcttccctgatacaggcagaccatctccggcgagcacggtcttgacagcgatggagtgtaagttcaataaccgactcgcagttccttgcgagagaccgcttccctgacggcttctcgggccagatgaaatgcaacagtactgacattctgccaatagctacaacggcaactctgagctccagctcgagcgcatgagcgtctacttcaacgaggtatgtcaccatgggcgacttcgggcttcacacattcggccagctactaactgaccacccacataacttaggcttccggcaacaagtacgttccccgcgccgtcctcgtcgatctcgagcccggtaccatggatgccgtccgtgctggtcccttcggccagctgttccgtcccgacaacttcgtcttcggtcagtccggtgctggcaacaattgggccaagggtcactacactgagggt

SEQ ID NO: 4 - DNA, 455 bp, Pythium aphanidermatum
  source 1..455: mol_type = genomic DNA
cttcagtgaactccatctcgtccataccctcaccagtgtaccagtgcaagaaggccttacgacggaacatggccgtgaactgctcgctgacacgcttgaacatctcctggatggcagtcgagttaccgatgaacgtggcgctcatcttgagaccctttggtgggatgtcacaaacgctggccttgatgttgttcgggatccactcaacgaagtacgacgagttcttgttctgaacgttgagcatctgctcgtcgacctccttggtgctcatacgaccacggaacatacaagcggcggtcaggtaacgaccgtgacgaggatcagcggcacacatcatgttcttggcgtcgaactgctgctgggtcagctctggcaccgtaagggcacggtactgctgcgagccgcgcgaggtgagcggagcgaaaccgaccatgaagaagtggaacgggggaaaa

SEQ ID NO: 5 - DNA, 518 bp, Gaeumannomyces graminis
  misc_feature 1..518: note = Gaeumannomyces graminis var. avenae
  source 1..518: mol_type = genomic DNA
ttagtgacccttggcccagttgttgccagcaccagactggccgaaaacgaagttgtcggggcggaacagctggccgaagggaccggcacgaacggcgtccatggtgccgggctcgagatcgacgaggacggcacgggggacatgcttgttgccggaggcctggagcggaaaggttatgggtcagaatacatgatacgaaggtgggaaataccggctgctaatgccggacagaagcttcaactcagggcctgtctgcatacctcgttgaagtagacgctcatgcgctcgagctggagctccgaggtgccgttgtacctgtatcaatatgtcagagcggtgaacggacggcgggccgagccacaagcaggacgaaatacgtacacgccattgctgtcgagaccgtgctcgctagaaatggtctgcctgtcaaagaagtcagtacgggtcacgggcagtggcagtcgtggtcggcggcggatcgtcgcgcggcgtcgtttcataccagaaagcagcaccgt

SEQ ID NO: 6 - DNA, 550 bp, Microdochium nivale
  misc_feature 1..550: note = Microdochium nivale var.
  source 1..550: mol_type = genomic DNA
ggtaaccaaatcggtgctgctttctggtgcgtacaactccgatactcaacgacggccgcagtgacctttgcgacgaaaacaaactcggcggtcaaacccgtatcgccgaaaatcttcggtcgcagaggaatctggcaaaagggtggaaataaacaagcaaggctaacactctcttccccgacacaggcaaaccatctccagtgagcacggtctcgacagcaatggcgtgtaagttcaataaccgactcgcacttcttgcgaaaggccacttccctgatggcgtatcacgccagatgaaatacacaagtactgacatcctgtcaatagctacaacggcacctccgagctccagctcgagcgcatgagtgtctacttcaatgaggcttccggcaacaagtacgttcctcgtgccgtccttgtcgatctcgagcccggtaccatggatgccgtccgtgctggtcccttcggccagctgttccgccccgacaacttcgtcttcggtcagtccggtgctggcaacaactgggccaagggtcactacactgagggt

SEQ ID NO: 7 - DNA, 485 bp, Magnaporthe poae
  source 1..485: mol_type = genomic DNA
ttagtgacccttggcccagttgttgccagcaccggactggccgaaaacgaagttgtcggggcggaacagctggccgaagggaccagcacggacagcatccatggtgccgggctcgagatcgaccaggacggcacgggggacatgcttgttgccggaggcctagagcgcggggaggcaatggtgtcagaaaaacaacacgtggttgcgaaagagagacgcgttcggagtctatctgcatacctcgttgaagtagacgctcatgcgctcgagctggagctccgaggtaccgttgtaactgcaccaatatgtcagagcggtgaacggacatgtggccgaggatctcccaaacagaatacatacactccattgctgtcgagaccgtgctcgctggagatggtttgcctgcccaggaagtcagtatcaatgatggatgatcacggtcgtggtgggtgcgagcggtggttcgtaccagaaagcagcaccgt

SEQ ID NO: 8 - DNA, 539 bp, Gaeumannomyces graminis
  misc_feature 1..539: note = Gaeumannomyces graminis var. avenae
  source 1..539: mol_type = genomic DNA
cctcagtgaactccatctcgtccataccctcgccagtgtaccaatgaaggaaagccttgcgcctgaacatggcagtgaactgctcaccaacacgcttgaagagctcttgtatggcagtcgagtttccgatgaaggtcgacgacatcttcaggccccggggagggattgagcagagggcggtctggatgttgttgggaatccactcgacgaagtacgacgagttcttgttctggatgttgcgcatctggtcctcgacctccttcatggagaccttaccacggctatcgcacacagggatggttagttagtgccttctaggttgggcatattaaatgggccagataaataagcccaatgcctagatgcaagactcacaaaatagcagagcaggtcaggtagcgaccgttgcggaagtccgaggcagccatcatgttcttggggtcgaacatctgctgggtcaactcgggcaccgtgacggcgcggaatgagtgggcgccgcggctagtcaggggagcgaagccgaccatgaagaagtggag

SEQ ID NO: 9 - DNA, 236 bp, Rhizoctonia solani
  misc_feature 1..236: note = Rhizoctonia solani AG2-2IV
  source 1..236: mol_type = genomic DNA
gttgtagggctcaacaaccgtgtcggagaccttgggggaaggaacgaccgagaatgtgcacatcatacgatcggggtattcttcacggatcttggagatcaaaagggtgcccataccggcaccggttcctccaccgagcgagtgggtaatctggaagccctgaagacactcgcatccctcggcctctttgcgcgcgacatcgagaactgcgtcaacaagctcggcaccttcggtgt

SEQ ID NO: 10 - DNA, 604 bp, Pythium aphanidermatum
  source 1..604: mol_type = genomic DNA
tgctttttcaggtgtagttggtacaactttatctgttttaattagaatggaattagcacaacctggtaatcaaatttttatgggaaatcatcaattatataatgttgttgtaacagcacatgcttttataatgattttcttcatggttatgcctgtattaattggtggttttggtaactggtttattcctttaatgattggtgctccagatatggcttttcctagaatgaataatattagtttttggttattacctccttcattattattattagtatcatctgctatagtagaatcaggtgctggtacaggttggactgtatatccaccattatcaagtgtacaagcacactcaggaccttcagtagatttagctatttttagtttacatttatctggtatttcttcattattaggtgctattaattttttatcaactatttataatatgagagctcctggattaagttttcatagattgccattatttgtttggtctgtttttattacagcttttttattattgttaacattaccagtattagcaggtgctattacaatgttattaacagatagaaatttaaatacttctttttatgatcct

SEQ ID NO: 11 - DNA, 657 bp, Colletotrichum graminicola
  source 1..657: mol_type = genomic DNA
aatattctcgacatatgcagcctttccgttgagatactatgtacgatcactgttagcatctcttttcaaaaaaggtcttgttggtgtccacgaacctgaaggtagtacgcgtgctcccacatgtcaataccaaagatgggcacgcccttggtgacagggtcctggtctttcgtcgtgataatgctgaggcccgttatgtcatccttaacaagccacccccagccgctaccggtgatacccagcagcgtggtgttgaaagcctgcttgaactggtcgagcccgccccagacgcgggtgatctcggcgacgagctttggcgccgcatcgggcgaggcatcaccgctcgaggctggggaaaggttctcccagaatagggaatggttgatgtggccgccgccgttgaagtttagggccgcgaggacggcgatgcgattctggagcgggtttgcattgtaagtctcgatggccttgttcagatttgtaacgtatgcttgatggctgtaggtggcttcatgtcaactctcttcttcgctgcttcatatttcatggttatctcactgtttgctgtggtgcagctccatgatctgagctgagatgtgaggctcgagggcctgcaggaggggtcagcgggcgcgatcgcgagcacgagtaagggat

SEQ ID NO: 12 - DNA, 663 bp, Colletotrichum cereale
  source 1..663: mol_type = genomic DNA
cgttccagatgttctcgacgtacgccgcttttccattgaggtactgaggccgagcattgttagtaccttccaacaaagcagatccgtcagtgtttacgaacctggaggtagtacgcgtgctcccacatgtccacgccgaagatgggcacgcccttggtgacagggtcctggtctttcgtcgtgatgatgctcagacccgttacgtcgtccttgaccagccatccccagccgctgccggtgatacccagaagcgtggcgttgaaagcctgcttgaactggtcgagcccgccccagacccgggcgatctcagcgacgagcttcggcgcggcgtctggcgaggcgtctgggctcgaggcaggggacaggttttcccagaagagggagtggttgatgtggccgccgccgttgaagttgagggctgggaggacggcgatgcggttctggagggggttcgcgttgtaggtctcgacggccttgtttagatttgtaacgtatgcttcgtgactgcgatggtttgatttcaaccctgttcttctttggtttctagtgcctagctctcttactgtttgctgtggtgcagctccatgatctgggctgagatgtgcggctcgagagcctggaagaggggtcagcgggtgcgaccgcgaacacaagtacggggat

SEQ ID NO: 13 - DNA, 703 bp, Pythium ultimum
  misc_feature 1..703: note = Pythium ultimum var. ultimum
  source 1..703: mol_type = genomic DNA
tcagaagaaaggtttcctacctcagacagcgtacgccatcctttactttcatttcgcgctggggtttccacaccctaacacttgcacacatgttagactccttggtccgtgtttcaagacgggccgaatcgctccatttcgtcaaagtcccgaacggcaaaagttactctagatctcaatcgaccaatcactccgtcagcatagcaagctatccaaacaggtaaccaaacgagagtcccaaacactttaaagcacattgtaggcacctcagtcccaaccacgacaactaactaccaagatataacagccaagagcaagctcctaacctacctcctcagtagccatttctcacagcatacgaactgactctgacgtcccaccgcaacacagggcaccaacaagcaaacgcagaacagcacaaagagcagaaaaccacttcttacatactgcacgcacctactcgccaatgaaatatgctacagattatagacactggatacgattcgcttccctttcagcagtttcaggtactctttaactctcttttcaaagttcttttcatctttccctcacggtacttgttcgctatcggtctcgcaccaatatttagctttagatggaatttaccacctactttgcgctgcagtcccaaacaacgcgactcaaagaaaacgtgtcgtacgcacaagctactcaggcacaa

SEQ ID NO: 14 - DNA, 18 bp, synthetic construct
  misc_feature 1..18: note = Primer for fungal detection
  source 1..18: mol_type = other DNA
gctcagccttcaacttgt

SEQ ID NO: 15 - DNA, 45 bp, synthetic construct
  misc_feature 1..45: note = Primer for fungal detection
  source 1..45: mol_type = other DNA
gcctttccagaatccctttgctttttgaaggaaccctttcccatc

SEQ ID NO: 16 - DNA, 19 bp, synthetic construct
  misc_feature 1..19: note = Primer for fungal detection
  source 1..19: mol_type = other DNA
ggtggaattgacaagcgta

SEQ ID NO: 17 - DNA, 43 bp, synthetic construct
  misc_feature 1..43: note = Primer for fungal detection
  source 1..43: mol_type = other DNA
atgctccgcgcatgagagttttcttctcatcgagatcagtgtc

SEQ ID NO: 18 - DNA, 21 bp, synthetic construct
  misc_feature 1..21: note = Primer for fungal detection
  source 1..21: mol_type = other DNA
ccgttaataggaagccaagga

SEQ ID NO: 19 - DNA, 19 bp, synthetic construct
  misc_feature 1..19: note = Primer for fungal detection
  source 1..19: mol_type = other DNA
tccatcagcaccaagatcg

SEQ ID NO: 20 - DNA, 20 bp, synthetic construct
  misc_feature 1..20: note = Primer for fungal detection
  source 1..20: mol_type = other DNA
atttaccttggccaaccttt

SEQ ID NO: 21 - DNA, 41 bp, synthetic construct
  misc_feature 1..41: note = Primer for fungal detection
  source 1..41: mol_type = other DNA
gcttcacacgctgctcctttttggagtcctcagcgatagat

SEQ ID NO: 22 - DNA, 19 bp, synthetic construct
  misc_feature 1..19: note = Primer for fungal detection
  source 1..19: mol_type = other DNA
tgtagctggctccattagt

SEQ ID NO: 23 - DNA, 41 bp, synthetic construct
  misc_feature 1..41: note = Primer for fungal detection
  source 1..41: mol_type = other DNA
ggggtaggggtcccaatcatttttgcacaccttttgctctt

SEQ ID NO: 24 - DNA, 22 bp, synthetic construct
  misc_feature 1..22: note = Primer for fungal detection
  source 1..22: mol_type = other DNA
tagctggatctcagtgttatgc

SEQ ID NO: 25 - DNA, 19 bp, synthetic construct
  misc_feature 1..19: note = Primer for fungal detection
  source 1..19: mol_type = other DNA
ctcccatccatgtctctgc

SEQ ID NO: 26 - DNA, 18 bp, synthetic construct
  misc_feature 1..18: note = Primer for fungal detection
  source 1..18: mol_type = other DNA
aatgtgtgaagcccgaag

SEQ ID NO: 27 - DNA, 42 bp, synthetic construct
  misc_feature 1..42: note = Primer for fungal detection
  source 1..42: mol_type = other DNA
gcttccctgacggcttctcttttctcagagttgccgttgtag

SEQ ID NO: 28 - DNA, 22 bp, synthetic construct
  misc_feature 1..22: note = Primer for fungal detection
  source 1..22: mol_type = other DNA
gtcgcagaggaatctaacataa

SEQ ID NO: 29 - DNA, 44 bp, synthetic construct
  misc_feature 1..44: note = Primer for fungal detection
  source 1..44: mol_type = other DNA
aggaactgcgagtcggttattgtttttacaggcagaccatctcc

SEQ ID NO: 30 - DNA, 22 bp, synthetic construct
  misc_feature 1..22: note = Primer for fungal detection
  source 1..22: mol_type = other DNA
acagtactgacattctgccaat

SEQ ID NO: 31 - DNA, 19 bp, synthetic construct
  misc_feature 1..19: note = Primer for fungal detection
  source 1..19: mol_type = other DNA
acactccatcgctgtcaag

SEQ ID NO: 32 - DNA, 20 bp, synthetic construct
  misc_feature 1..20: note = Primer for fungal detection
  source 1..20: mol_type = other DNA
tgaactccatctcgtccata

SEQ ID NO: 33 - DNA, 18 bp, synthetic construct
  misc_feature 1..18: note = Primer for fungal detection
  source 1..18: mol_type = other DNA
tcgtatgagcaccaagga

SEQ ID NO: 34 - DNA, 19 bp, synthetic construct
  misc_feature 1..19: note = Primer for fungal detection
  source 1..19: mol_type = other DNA
gatgttcaagcgtgtcagc

SEQ ID NO: 35 - DNA, 19 bp, synthetic construct
  misc_feature 1..19: note = Primer for fungal detection
  source 1..19: mol_type = other DNA
gctggccttgatgttgttc

SEQ ID NO: 36 - DNA, 42 bp, synthetic construct
  misc_feature 1..42: note = Primer for fungal detection
  source 1..42: mol_type = other DNA
cggtaactcgactgccatccttttccttacgacggaacatgg

SEQ ID NO: 37 - DNA, 44 bp, synthetic construct
  misc_feature 1..44: note = Primer for fungal detection
  source 1..44: mol_type = other DNA
ccctttggtgggatgtcacaattttctcgtcgtacttcgttgag

SEQ ID NO: 38 - DNA, 19 bp, synthetic construct
  misc_feature 1..19: note = Primer for fungal detection
  source 1..19: mol_type = other DNA
ttgaagtagacgctcatgc

SEQ ID NO: 39 - DNA, 19 bp, synthetic construct
  misc_feature 1..19: note = Primer for fungal detection
  source 1..19: mol_type = other DNA
gtgctgctttctggtatga

SEQ ID NO: 40 - DNA, 18 bp, synthetic construct
  misc_feature 1..18: note = Primer for fungal detection
  source 1..18: mol_type = other DNA
cgttcaccgctctgacat

SEQ ID NO: 41 - DNA, 19 bp, synthetic construct
  misc_feature 1..19: note = Primer for fungal detection
  source 1..19: mol_type = other DNA
cgctagaaatggtctgcct

SEQ ID NO: 42 - DNA, 43 bp, synthetic construct
  misc_feature 1..43: note = Primer for fungal detection
  source 1..43: mol_type = other DNA
ttcgtcctgcttgtggctcttttgccgttgtacctgtatcaat

SEQ ID NO: 43 - DNA, 41 bp, synthetic construct
  misc_feature 1..41: note = Primer for fungal detection
  source 1..41: mol_type = other DNA
ttgctgtcgagaccgtgctttttgacccgtactgacttctt

SEQ ID NO: 44 - DNA, 18 bp, synthetic construct
  misc_feature 1..18: note = Primer for fungal detection
  source 1..18: mol_type = other DNA
ggtaaccaaatcggtgct

SEQ ID NO: 45 - DNA, 19 bp, synthetic construct
  misc_feature 1..19: note = Primer for fungal detection
  source 1..19: mol_type = other DNA
gcgagtcggttattgaact

SEQ ID NO: 46 - DNA, 19 bp, synthetic construct
  misc_feature 1..19: note = Primer for fungal detection
  source 1..19: mol_type = other DNA
tttcgtcgcaaaggtcact

SEQ ID NO: 47 - DNA, 21 bp, synthetic construct
  misc_feature 1..21: note = Primer for fungal detection
  source 1..21: mol_type = other DNA
aacaagcaaggctaacactct

SEQ ID NO: 48 - DNA, 41 bp, synthetic construct
  misc_feature 1..41: note = Primer for fungal detection
  source 1..41: mol_type = other DNA
gggtttgaccgccgagttttttgcgtacaactccgatactc

SEQ ID NO: 49 - DNA, 43 bp, synthetic construct
  misc_feature 1..43: note = Primer for fungal detection
  source 1..43: mol_type = other DNA
ggaatctggcaaaagggtggatttttgctcactggagatggtt

SEQ ID NO: 50 - DNA, 19 bp, synthetic construct
  misc_feature 1..19: note = Primer for fungal detection
  source 1..19: mol_type = other DNA
ttgaagtagacgctcatgc

SEQ ID NO: 51 - DNA, 18 bp, synthetic construct
  misc_feature 1..18: note = Primer for fungal detection
  source 1..18: mol_type = other DNA
gctgctttctggtacgaa

SEQ ID NO: 52 - DNA, 20 bp, synthetic construct
  misc_feature 1..20: note = Primer for fungal detection
  source 1..20: mol_type = other DNA
tcaccgctctgacatattgg

SEQ ID NO: 53 - DNA, 18 bp, synthetic construct
  misc_feature 1..18: note = Primer for fungal detection
  source 1..18: mol_type = other DNA
gctggagatggtttgcct

SEQ ID NO: 54 - DNA, 42 bp, synthetic construct
  misc_feature 1..42: note = Primer for fungal detection
  source 1..42: mol_type = other DNA
ggagatcctcggccacatgttttcgaggtaccgttgtaactg

SEQ ID NO: 55 - DNA, 42 bp, synthetic construct
  misc_feature 1..42: note = Primer for fungal detection
  source 1..42: mol_type = other DNA
ttgctgtcgagaccgtgcttttaccgtgatcatccatcattg

SEQ ID NO: 56 - DNA, 19 bp, synthetic construct
  misc_feature 1..19: note = Primer for fungal detection
  source 1..19: mol_type = other DNA
gacgaagtacgacgagttc

SEQ ID NO: 57 - DNA, 19 bp, synthetic construct
  misc_feature 1..19: note = Primer for fungal detection
  source 1..19: mol_type = other DNA
ctccacttcttcatggtcg

SEQ ID NO: 58 - DNA, 19 bp, synthetic construct
  misc_feature 1..19: note = Primer for fungal detection
  source 1..19: mol_type = other DNA
cgatagccgtggtaaggtc

SEQ ID NO: 59 - DNA, 19 bp, synthetic construct
  misc_feature 1..19: note = Primer for fungal detection
  source 1..19: mol_type = other DNA
aggcagccatcatgttctt

SEQ ID NO: 60 - DNA, 44 bp, synthetic construct
  misc_feature 1..44: note = Primer for fungal detection
  source 1..44: mol_type = other DNA
gcccaacctagaaggcactaactttttcctcgacctccttcatg

SEQ ID NO: 61 - DNA, 41 bp, synthetic construct
  misc_feature 1..41: note = Primer for fungal detection
  source 1..41: mol_type = other DNA
ggtcaggtagcgaccgttgttttgagttgacccagcagatg

SEQ ID NO: 62 - DNA, 18 bp, synthetic construct
  misc_feature 1..18: note = Primer for fungal detection
  source 1..18: mol_type = other DNA
ttgtagggctcaacaacc

SEQ ID NO: 63 - DNA, 18 bp, synthetic construct
  misc_feature 1..18: note = Primer for fungal detection
  source 1..18: mol_type = other DNA
gacgcagttctcgatgtc

SEQ ID NO: 64 - DNA, 44 bp, synthetic construct
  misc_feature 1..44: note = Primer for fungal detection
  source 1..44: mol_type = other DNA
ggcacccttttgatctccaagattttggaacgaccgagaatgtg

SEQ ID NO: 65 - DNA, 40 bp, synthetic construct
  misc_feature 1..40: note = Primer for fungal detection
  source 1..40: mol_type = other DNA
ataccggcaccggttcctttttgatgcgagtgtcttcagg

SEQ ID NO: 66 - DNA, 20 bp, synthetic construct
  misc_feature 1..20: note = Primer for fungal detection
  source 1..20: mol_type = other DNA
ccgtgaagaataccccgatc

SEQ ID NO: 67 - DNA, 18 bp, synthetic construct
  misc_feature 1..18: note = Primer for fungal detection
  source 1..18: mol_type = other DNA
ccgagcgagtgggtaatc

SEQ ID NO: 68 - DNA, 22 bp, synthetic construct
  misc_feature 1..22: note = Primer for fungal detection
  source 1..22: mol_type = other DNA
aattagcacaacctggtaatca

SEQ ID NO: 69 - DNA, 22 bp, synthetic construct
  misc_feature 1..22: note = Primer for fungal detection
  source 1..22: mol_type = other DNA
acttaatccaggagctctcata

SEQ ID NO: 70 - DNA, 48 bp, synthetic construct
  misc_feature 1..48: note = Primer for fungal detection
  source 1..48: mol_type = other DNA
gccatatctggagcaccaatcattttaatgttgttgtaacagcacatg

SEQ ID NO: 71 - DNA, 47 bp, synthetic construct
  misc_feature 1..47: note = Primer for fungal detection
  source 1..47: mol_type = other DNA
aatcaggtgctggtacaggttgttttaatctactgaaggtcctgagt

SEQ ID NO: 72 - DNA, 22 bp, synthetic construct
  misc_feature 1..22: note = Primer for fungal detection
  source 1..22: mol_type = other DNA
aaccaccaattaatacaggcat

SEQ ID NO: 73 - DNA, 22 bp, synthetic construct
  misc_feature 1..22: note = Primer for fungal detection
  source 1..22: mol_type = other DNA
accattatcaagtgtacaagca

SEQ ID NO: 74 - DNA, 21 bp, synthetic construct
  misc_feature 1..21: note = Primer for fungal detection
  source 1..21: mol_type = other DNA
tgtacgatcactgttagcatc

SEQ ID NO: 75 - DNA, 18 bp, synthetic construct
  misc_feature 1..18: note = Primer for fungal detection
  source 1..18: mol_type = other DNA
ctcgaccagttcaagcag

SEQ ID NO: 76 - DNA, 41 bp, synthetic construct
  misc_feature 1..41: note = Primer for fungal detection
  source 1..41: mol_type = other DNA
ggcgtgcccatctttggtattttgtgtccacgaacctgaag

SEQ ID NO: 77 - DNA, 43 bp, synthetic construct
  misc_feature 1..43: note = Primer for fungal detection
  source 1..43: mol_type = other DNA
ggtgacagggtcctggtctttttgtggcttgttaaggatgaca

SEQ ID NO: 78 - DNA, 18 bp, synthetic construct
  misc_feature 1..18: note = Primer for fungal detection
  source 1..18: mol_type = other DNA
ttgacatgtgggagcacg

SEQ ID NO: 79 - DNA, 20 bp, synthetic construct
  misc_feature 1..20: note = Primer for fungal detection
  source 1..20: mol_type = other DNA
tcgtcgtgataatgctgagg

SEQ ID NO: 80 - DNA, 19 bp, synthetic construct
  misc_feature 1..19: note = Primer for fungal detection
  source 1..19: mol_type = other DNA
cgttccagatgttctcgac

SEQ ID NO: 81 - DNA, 18 bp, synthetic construct
  misc_feature 1..18: note = Primer for fungal detection
  source 1..18: mol_type = other DNA
gtcaaggacgacgtaacg

SEQ ID NO: 82 - DNA, 47 bp, synthetic construct
  misc_feature 1..47: note = Primer for fungal detection
  source 1..47: mol_type = other DNA
gcgtactacctccaggttcgtattttgagcattgttagtaccttcca

SEQ ID NO: 83 - DNA, 41 bp, synthetic construct
  misc_feature 1..41: note = Primer for fungal detection
  source 1..41: mol_type = other DNA
atgtccacgccgaagatggttttctgagcatcatcacgacg

SEQ ID NO: 84 - DNA, 19 bp, synthetic construct
  misc_feature 1..19: note = Primer for fungal detection
  source 1..19: mol_type = other DNA
aacactgacggatctgctt

SEQ ID NO: 85 - DNA, 18 bp, synthetic construct
  misc_feature 1..18: note = Primer for fungal detection
  source 1..18: mol_type = other DNA
tgacagggtcctggtctt

SEQ ID NO: 86 - DNA, 18 bp, synthetic construct
  misc_feature 1..18: note = Primer for fungal detection
  source 1..18: mol_type = other DNA
caaacgagagtcccaaac

SEQ ID NO: 87 - DNA, 22 bp, synthetic construct
  misc_feature 1..22: note = Primer for fungal detection
  source 1..22: mol_type = other DNA
atccagtgtctataatctgtag

SEQ ID NO: 88 - DNA, 44 bp, synthetic construct
  misc_feature 1..44: note = Primer for fungal detection
  source 1..44: mol_type = other DNA
gagtcagttcgtatgctgtgagtttttaacagccaagagcaagc

SEQ ID NO: 89 - DNA, 44 bp, synthetic construct
  misc_feature 1..44: note = Primer for fungal detection
  source 1..44: mol_type = other DNA
cgcaacacagggcaccaacttttgtgcagtatgtaagaagtggt

SEQ ID NO: 90 - DNA, 19 bp, synthetic construct
  misc_feature 1..19: note = Primer for fungal detection
  source 1..19: mol_type = other DNA
ggctactgaggaggtaggt

SEQ ID NO: 91 - DNA, 20 bp, synthetic construct
  misc_feature 1..20: note = Primer for fungal detection
  source 1..20: mol_type = other DNA
agaacagcacaaagagcaga

(19) United States
(12) Patent Application Publication
(10) Pub. No.: US 2026/0226565 A1
(43) Pub. Date: Aug. 6, 2026

(54) METHODS FOR DETECTING FUNGI IN TURF GRASS WITH A LAMP ASSAY HAVING NOVEL PRIMER SETS

(71) Applicant: SYNGENTA CROP PROTECTION AG, Basel (CH)

(72) Inventors: Marilena PALMISANO, Wädenswil (CH); Christian WOHLER, Wädenswil (CH)

(73) Assignee: SYNGENTA CROP PROTECTION AG, Basel (CH)

(21) Appl. No.: 19/467,176

(22) Filed: Feb. 2, 2026

Related U.S. Application Data
(62) Division of application No. 18/185,738, filed on Mar. 17, 2023, now Pat. No. 12,545,966.
Application No. 18/185,738 is a division of application No. 16/967,319, filed on Feb. 5, 2019, now Pat. No. 11,634,783.

(30) Foreign Application Priority Data
Feb. 5, 2018 (EP) 18155093.0
Mar. 2, 2018 (EP) 18159821.0

(51) Int. Cl. C12Q 1/6895

(52) U.S. Cl. CPC C12Q 1/6895; C12Q 2600/16

(57) ABSTRACT

The present invention provides a method for detecting fungal DNA in a turf grass sample with a loop-mediated isothermal amplification (LAMP) assay which contains primers for fungal DNA of at least one turf pathogenic fungi selected from Sclerotinia homoeocarpa, Rhizoctonia solani spp., Pythium aphanidermatum, Gaeumannomyces graminis spp., Microdochium nivale spp., Magnaporthe poae, Colletotrichum graminicola, Colletotrichum cereale and Pythium ultimum var. ultimum, comprising: subjecting the turf sample to a LAMP reaction wherein the LAMP reaction uses a primer set of four or more nucleic acid sequences with each primer in the set having from 15 to 50 nucleic acids The primers useful in the present method are selected from specifically selected internal transcribed spacer regions or genes of the target fungi to provide improved assay results.

### CROSS-REFERENCE TO RELATED APPLICATIONS

This application is a divisional of U.S. application Ser. No. 18/185,738, filed Mar. 17, 2023, which is a divisional of U.S. application Ser. No. 16/967,319, filed Aug. 4, 2020, which is a 371 National Stage application of International Application No. PCT/EP2019/052803, filed Feb. 5, 2019, which claims priority to EP 18159821.0, filed Mar. 2, 2018, and EP 18155093.0, filed Feb. 5, 2018, the entire contents of which are incorporated by reference herein.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 23, 2026, is named “115479.001111_sequence-xml” and is 122,491 bytes in size. The sequence listing contained in this .XML file is part of the specification and is hereby incorporated by reference herein in its entirety.

The present invention relates to a method of detecting diseases in turf grass caused by fungal pathogens with a loop-mediated isothermal amplification (LAMP) assay of a sample of such turf grass to detect nucleic acids from one or more fungi.

LAMP or Loop-mediated Isothermal Amplification as described in e.g. U.S. Pat. No. 6,410,278 (Eiken) is a DNA amplification method characterized by the use of at least 4 or more different primers that are specifically designed to recognize 6 distinct regions on the target gene and the reaction process proceeds at a constant temperature using strand displacement reaction. Amplification and detection of target nucleic acid of interest can be completed in a single step, by incubating the mixture of the biological sample or a nucleic acid extract thereof, primers, DNA polymerase with strand displacement activity and substrates at a constant temperature (about 65′C). It provides high amplification efficiency, with DNA being amplified numerous times in 15-60 minutes. Because of its high specificity, the presence of amplified product can indicate the presence of target gene.

There are numerous problems that turf grass managers face in maintaining turf grass at a standard of quality expected by users. While the problems are many, those relating to disease (including diseases caused by fungal pathogens) are particularly challenging to manage and control. For example, disease can affect turf grass plants on golf courses causing a loss of revenue from reduced quality including playability. One example of a common problem for golf course managers is knowing which disease is present so that appropriate and timely management techniques can be taken. Relevant turf diseases caused by turf pathogenic microorganisms include, for example, anthracnose, take-all patch, summer patch, snow mold, pythium blight, brown patch and dollar spot.

Agricultural active chemicals for controlling pathogens, such as fungicides, are typically applied on golf courses as needed depending on the extent of disease pressure, pathogen population, weather, and the like. However, fungicide applications are highly controlled by course budget, availability of appropriate equipment, and availability of qualified personnel for applying the agricultural active chemicals.

In view of these problems, a rapid and reliable assay for detection of turf pathogenic fungi would be extremely useful. Known PCR assays are not practical to use in golf course or other intensively managed turf grass or professional landscape settings, as PCR requires specialised laboratory skills and instruments. Certain other molecular biology methods for decting fungal disease in turf grass are known and described, for example, in WO2009147017 which relates to a TRFLP methodology.

The present invention accordingly relates to a LAMP assay for detecting the presence of DNA in a turf sample which is associated with selected fungal pathogens that cause relevant turf diseases including, for example, anthracnose, take-all patch, summer patch, snow mold, pythium blight, brown patch and dollar spot.

To facilitate timely and efficient detection of turf grass disease pathogens and to improve the cost and effectiveness of turf grass disease treatments, a LAMP assay according to the invention can be utilized to earlier detect DNA associated with fungal pathogens which cause relevant turf diseases. In accordance with the invention, the LAMP method suitably uses a primer set of at least four and preferably six or more nucleic acid sequences derived from the target disease pathogens. More particularly, the inventive method provides that each primer used in the selected primer set for the LAMP assay has from 15 to 50 nucleic acids and where the primers in the set are selected from a specific DNA loci within the target fungi.

In accordance with the present invention, a method for detecting fungal DNA in a turf grass sample with a loop-mediated isothermal amplification (LAMP) assay is provided which contains primers for fungal DNA (nucleic acids) of a turf pathogenic fungi selected from the group consisting of Sclerotinia homoeocarpa, Rhizoctonia solani spp., Pythium aphanidermatum, Gaeumannomyces graminis spp., Microdochium nivale spp., Magnaporthe poae, Colletotrichum graminicola, Colletotrichum cereale and Pythium ultimum var. ultimum(target fungi). The LAMP assay of the present invention uses a primer set of at least four and preferably six or more nucleic acid sequences with each primer in the set having from 15 to 50 nucleic acids, and where the fungal DNA to be detected is obtained from a target fungal pathogen. The primers useful in the present LAMP assay method are selected from specific internal transcribed spacer regions or genes of the target fungi to provide improved assay results.

In a particular embodiment, the Microdochium nivale spp. target fungi are selected from Microdochium nivale var. nivale and Microdochium nivale var. majus. In another embodiment, the Gaeumannomyces graminis spp. target fungi are selected from Gaeumannomyces graminis var. avenge, Gaeumannomyces graminis var. graminis and Gaeumannomyces graminis var. tritici. In a further embodiment, the Rhizoctonia solani spp. target fungi are selected from Rhizoctonia solani AG2-21V and Rhizoctonia solani AG2-2111B.

In the context of the present invention, detection of fungal DNA with the inventive LAMP assay in a turf sample may be indicative of the presence of fungal pathogens and can also assist in assesing the onset or presence of a turf disease condition as follows:

| Fungal Pathogen | Turf Disease |
|---|---|
| Sclerotinia homoeocarpa | Dollar Spot |
| Rhizoctonia solani spp. | Brown Patch |
| Microdochium nivale spp. | Snow Mold |
| Pythium aphanidermatum | Pythium Blight |
| Gaeumannomyces graminis spp. | Take-all patch |
| Magnaporthe poae | Summer patch |
| Colletotrichum graminicola | Anthracnose |
| Colletotrichum cereale | Anthracnose |
| Pythium ultimum | Pythium Blight |

In one embodiment,
(a) the primer set for Sclerotinia homoeocarpa DNA is selected from within the DNA of SEQ ID NO: 1; (b) the primer set for Rhizoctonia solani DNA is selected from within the DNA of SEQ ID NO: 2 or SEQ ID NO: 9;
(c) the primer set for Microdochium nivale spp. DNA (preferably Microdochium nivale var. nivale) is selected from within the DNA of SEQ ID NO: 3;
(d) the primer set for Pythium aphanidermatum DNA is selected from within the DNA of SEQ ID NO: 4 or SEQ ID NO: 10;
(e) the primer set for Gaeumannomyces graminis spp. DNA (preferably Gaeumannomyces graminis var. avenae, Gaeumannomyces graminis var. graminis or Gaeumannomyces graminis var. tritici) is selected from within the DNA of SEQ ID NO: 5 or SEQ ID NO: 8;
(f) the primer set for Microdochium nivale spp. DNA (preferably Microdochium nivale var. majus) is selected from within the DNA of SEQ ID NO:6;
(g) the primer set for Magnaporthe poae DNA is selected from within the DNA of SEQ ID NO: 7;
(h) the primer set for Colletotrichum graminicola DNA is selected from within the DNA of SEQ ID NO: 11;
(i) the primer set for Colletotrichum cereale DNA is selected from within the DNA of SEQ ID NO: 12; and
(j) the primer set for Pythium ultimum var. Ultimum DNA is selected from within the DNA of SEQ ID NO: 13

Preferably the LAMP primer sets sutiable for use in detecting fungi DNA in turf samples according to the present invention comprise four primers including: a pair of forward (FIP) and reverse (BIP) inner primers, and a pair of forward (F3) and reverse (B3) outer primers. More preferably, the LAMP primer sets sutiable for use in the present invention include the addition of loop forward (LF) and/or loop back (LB) primers to accelerate amplification of nucleic acid present in the turf sample and to reduce the detection time of any target fungi that may be present in such turf sample. The LAMP primer set embodiments listed below relate to the detection of the target fungi DNA in turf samples in accordance with the method of the invention.

In the description of the embodiments which follow that are associated with the primers of SEQ ID Nos. 14-91 according to the invention, it will be understood that the primers useful in the present invention each independently and respectively have a sequence which is at least 90%, preferably at least 95%, more preferably at least 96%, and even more preferably at least 97% identical to the primers of SEQ IDs of 14-91.

In a particularly prefered embodiment, the primers useful in the present invention each independently and respectively have a sequence which is at least at least 98%, more preferably at least 99% identical to the primers of SEQ IDs of 14-91. Most preferably, the primers useful in the present invention each independently and respectively have a sequence which is identical to SEQ IDs of 14-91.

Accordingly, the present invention provides a method for detecting fungal DNA in a turf grass sample with a loop-mediated isothermal amplification (LAMP) assay which contains primers for fungal DNA of at least one turf pathogenic fungi selected from Sclerotinia homoeocarpa, Rhizoctonia solani spp., Pythium aphanidermatum, Gaeumannomyces graminis spp., Microdochium nivale spp., Magnaporthe poae, Colletotrichum graminicola, Colletotrichum cereale and Pythium ultimum var. ultimum, comprising: subjecting the turf sample to a LAMP reaction wherein the LAMP reaction uses a primer set of four or more nucleic acid sequences with each primer in the set having from 15 to 50 nucleic acids, and wherein the set of primers comprises at least one primer set as described below.

In one embodiment, the primer set for detecting Sclerotinia homoeocarpa DNA comprises or is selected from SEQ ID NOs: 15 and 27.

In another embodiment, the primer set for detecting Sclerotinia homoeocarpa DNA comprises or is selected from SEQ ID NOs:14, 15, 16 and 17.

In a further embodiment, the primer set for detecting Sclerotinia homoeocarpa DNA comprises or is selected from SEQ ID NOs: 14, 15, 16, 17, 18 and 19.

In a further embodiment, the primer set for detecting Rhizoctonia solani DNA comprises or is selected from SEQ ID NO: 23.

In another embodiment, the primer set for detecting Rhizoctonia solani DNA comprises or is selected from SEQ ID Nos: 63, 64 and 65.

In another embodiment, the primer set for detecting Rhizoctonia solani DNA comprises or is selected from SEQ ID NO: 20, 21, 22 and 23.

In another embodiment, the primer set for detecting Rhizoctonia solani DNA comprises or is selected from SEQ ID NO: 62, 63. 66 and 67.

In yet another embodiment, the primer set for detecting Rhizoctonia solani DNA comprises or is selected from SEQ ID Nos: 62, 63, 64, 65, 66 and 67.

In another embodiment, the primer set for detecting Rhizoctonia solani DNA comprises or is selected from SEQ ID Nos: 20, 21, 22, 23, 24 and 25.

In another embodiment, the primer set for detecting Microdochium nivale spp. (preferably Microdochium nivale var. nivale) DNA comprises or is selected from SEQ ID Nos: 27, 28 and 29.

In another embodiment, the primer set for detecting Microdochium nivale spp. (preferably Microdochium nivale var. nivale) DNA comprises or is selected from SEQ ID NO: 26, 27, 28, and 29.

In a further embodiment, the primer set for detecting Microdochium nivale spp. (preferably Microdochium nivale var. nivale) DNA comprises or is selected from SEQ ID Nos: 26, 27, 28, 29, 30 and 31.

In one embodiment, the primer set for detecting Pythium aphanidermatum DNA comprises or is selected from SEQ ID NOs:33, 36 and 37.

In another embodiment, the primer set for detecting Pythium aphanidermatum DNA comprises or is selected from SEQ ID NO: 32, 33, 36, and 37.

In another embodiment, the primer set for detecting Pythium aphanidermatum DNA comprises or is selected from SEQ ID NOs: 32, 33, 34, 35, 36 and 37.

In another embodiment, the primer set for detecting Pythium aphanidermatum DNA comprises or is selected from SEQ ID NOs:69, 70 and 71.

In another embodiment, the primer set for detecting Pythium aphanidermatum DNA comprises or is selected from SEQ ID NO: 68, 69, 72 and 73.

In another embodiment, the primer set for detecting Pythium aphanidermatum DNA comprises or is selected from SEQ ID NOs: 68, 69, 70, 71, 72 and 73.

In one embodiment, the primer set for detecting Gaeumannomyces graminis spp. (preferably Gaeumannomyces graminis var. avenae, Gaeumannomyces graminis var. graminis or Gaeumannomyces graminis var. tritici) DNA comprises or is selected from SEQ ID NO: 60.

In another embodiment, the primer set for detecting Gaeumannomyces graminis spp. (preferably Gaeumannomyces graminis var. avenae, Gaeumannomyces graminis var. graminis or Gaeumannomyces graminis var. tritici) DNA comprises or is selected from SEQ ID NOs: 42 and 43.

In a further embodiment, the primer set for detecting Gaeumannomyces graminis spp. (preferably Gaeumannomyces graminis var. avenae, Gaeumannomyces graminis var. graminis or Gaeumannomyces graminis var. tritici) DNA comprises or is selected from SEQ ID NO: 38, 39, 42 and 43.

In a further embodiment, the primer set for detecting Gaeumannomyces graminis spp. (preferably Gaeumannomyces graminis var. avenae, Gaeumannomyces graminis var. graminis or Gaeumannomyces graminis var. tritici) DNA comprises or is selected from SEQ ID NO: 56, 57, 60 and 61.

In another embodiment, the primer set for detecting Gaeumannomyces graminis spp. (preferably Gaeumannomyces graminis var. avenae, Gaeumannomyces graminis var. graminis or Gaeumannomyces graminis var. tritici) DNA comprises or is selected from SEQ ID NOs: 38, 39, 40, 41, 42 and 43.

In another embodiment, the primer set for detecting Gaeumannomyces graminis spp. (preferably Gaeumannomyces graminis var. avenae, Gaeumannomyces graminis var. graminis or Gaeumannomyces graminis var. tritici) DNA comprises or is selected from SEQ ID NO: 56, 57, 58, 59, 60 and 61.

In one embodiment, the primer set for detecting Microdochium nivale spp. (preferably Microdochium nivale var. majus) DNA comprises or is selected from SEQ ID Nos: 48 and 49.

In a further embodiment, the primer set for detecting Microdochium nivale spp. (preferably Microdochium nivale var. majus) DNA comprises or is selected from SEQ ID NO: 44, 45, 48 and 49.

In yet another embodiment, the primer set for detecting Microdochium nivale spp. (preferably Microdochium nivale var. majus) DNA comprises or is selected from SEQ ID Nos: 44, 45, 46, 47, 48 and 49.

In another embodiment, the primer set for detecting Magnaporthe poae DNA comprises or is selected from SEQ ID NOs: 54 and 55.

In a further embodiment, the primer set for detecting Magnaporthe poae DNA comprises or is selected from SEQ ID NO: 50, 51, 54 and 55.

In another embodiment, the primer set for detecting Magnaporthe poae DNA comprises or is selected from SEQ ID NOs: 50, 51, 52, 53, 54, and 55.

In one embodiment, the primer set for detecting Colletotrichum graminicola DNA comprises or is selected from SEQ ID NOs:74, 76 and 77.

In a further embodiment, the primer set for detecting Colletotrichum graminicola DNA comprises or is selected from SEQ ID NO: 74 75, 78 and 79.

In another embodiment, the primer set for detecting Colletotrichum graminicola DNA comprises or is selected from SEQ ID NOs:74, 75, 76, 77, 78 and 79.

In another embodiment, the primer set for detecting Colletotrichum cereale DNA comprises or is selected from SEQ ID Nos 80, 82 and 83.

In a further embodiment, the primer set for detecting Colletotrichum cereale DNA comprises or is selected from SEQ ID NO: 80, 81, 84 and 85.

In another embodiment, the primer set for detecting Colletotrichum cereale DNA comprises or is selected from SEQ ID Nos 80, 81, 82, 83, 84 and 85.

In one embodiment, the primer set for detecting Pythium ultimum var. Ultimum Idin-rc DNA comprises or is selected from SEQ ID Nos: 86, 88 and 89.

In a further embodiment, the primer set for detecting Pythium ultimum var. Ultimum Idin-rc DNA comprises or is selected from SEQ ID NO: 86, 87, 90 and 91.

In another embodiment, the primer set for detecting Pythium ultimum var. Ultimum Idin-rc DNA comprises or is selected from SEQ ID Nos: 86, 87, 88, 89, 90 and 91.

The LAMP assays of the invention can be used for detection, including early detection, of DNA from turf fungi selected from the group consisting of Sclerotinia homoeocarpa, Rhizoctonia solani, Pythium aphanidermatum, Gaeumannomyces graminis spp., Microdochium nivale spp., Magnaporthe poae, Colletotrichum graminicola, Colletotrichum cereale and Pythium ultimum var. Ultimum in turf samples which is easy to obtain and allows management and/or maintenance of the turf grass to be tailored accordingly.

According to the invention, by “turf grass” there is understood an annual or perennial Gramineae. Said gramineae preferably belongs to one or more of the genera Agropyron, Agrostis, Axonopus, Bromus, Buchlos, Cynodon, Eremochloa, Festuca, Lolium, Paspulum, Pennisetum, Phleum, Poa, Stenotaphrum or Zoysia. More preferably, said gramineae belongs to one or more of the genera Agrostis, Buchlos, Cynodon, Eremochloa, Festuca, Lolium, Paspulum, Pennisetum, Poa, Stenotaphrum or Zoysia.

In one embodiment, according to the invention by “turf” is understood as a group of turf grass, which covers a surface area of ground and is subject to regular maintenance.

The present invention can be practiced with all turf grasses, including cool season turf grass and warm season turf grass.

Examples of cool season turf grasses are: Bluegrasses (Poa L.), such as Kentucky Bluegrass (Poa pratensis L.), Rough Bluegrass (Poa trivialis L.), Canada Bluegrass (Poa compressa L.) and Annual Bluegrass (Poa annua L.); Bentgrasses (Agrostis L.), such as Creeping Bentgrass (Agrostis palustris Huds.), Colonial Bentgrass (Agrostis tenius Sibth.), Velvet Bentgrass (Agrostis canina L.) and Redtop (Agrostis alba L.); Fescues (Festuca L.), such as Creeping Red Fescue (Festuca rubra L.), Chewings Fescue (Festuca rubra var. commutata Gaud.), Sheep Fescue (Festuca ovina L.), Hard Fescue (Festuca longifolia), Tall Fescue (Festuca arundinacea Schreb.), Meadow Fescue (Festuca elatior L.); Ryegrasses (Lolium L.), such as Perennial Ryegrass (Lolium perenne L.), Annual (Italian) Ryegrass (Lolium multiflorum Lam.); Wheatgrasses (Agropyron Gaertn.), such as Fairway Wheatgrass (Agropyron cristatum (L.) Gaertn.), Western Wheatgrass (Agropyron smithii Rydb.). Other cool season turf grasses include Smooth Brome (Bromus inermis Leyss.) and Timothy Phleum L.).

Examples of warm season turf grasses are Bermudagrasses (Cynodon L. C. Rich), Zoysiagrasses Zoysia Willd.), St. Augustinegrass (Stenotaphrum secundatum (Walt.) Kuntze), Centipedegrass (Eremochloa ophiuroides (Munro.) Hack.), Carpetgrass (Axonopus Beauv.), Bahiagrass (Paspalum notatum Flugge.), Kikuyugrass (Pennisetum clandestinum Hochst. ex Chiov.), Buffalograss (Buchloe dactyloides (Nutt.) Engelm.) and Seashore paspalum (Paspalum vaginatum swartz).

The LAMP method invention also contemplates a kit for the detection of fungi in a turf grass sample using a LAMP assay. A test strip containing one or more than one of the primer sets as described herein can be utilized. In one embodiment, multiple primer sets are multiplexed on a test strip for the detection of multiple diseases from turf grass samples collected from a particular locus.

For example, a bijou tube with a ball bearing and a suitable amount of lysis buffer is provided with a 1 cubic cm homogenized turf sample and shaken vigorously for 1 minute. A test strip with sample well containing all the resuspension buffer and drops of this test solution are placed into a sample wells on a test strip wherein the wells have all the ingredients necessary to perform a LAMP reaction (e.g., the primer sets and a reagent such as an isothermal master mix cat no. iso-001 available from Optigene). In one embodiment, the test strips are multiplexed. In another embodiment, the test strip includes 8 wells, two control and 6 for turf diseases of interest. In one embodiment, the test strip is associated with a diagnostic instrument such as a Genie®II or III available from OptiGene.

### Primer Design

Highly conserved genes were used for the design of the LAMP primers for the detection of DNA from selected turf grass pathogens (column 1 of TABLE 3). Pure genomic DNA from all fungi of interest was obtained using the NucleoSpin Plant II (MACHEREY-NAGEL). With PCR technology the sequence of interest were amplified using published primer pairs followed by a Sanger sequencing. The following DNA Loci (genes and regions) were sequenced: Internal transcribed spacer (ITS), elongation factor 1-alpha (EF), beta-tubulin (Tub), cytochrome c oxidase subunit 1 (Cox), superoxide dismutase (SOD1) and large subunit nuclear ribosomal RNA (LSU). The raw sequences were aligned using ClustalW alignment method (CLC Main Workbench Software). The BLAST comparisons with sequences from GenBank (NCBI) were used to identify gene homologs. Ideally, a good sequence is defined by successful PCR amplification for all target taxa and no homology with other taxa.

The best sequences (SEQ ID Nos, 1-13) from the sequenced DNA Loci were then used for the design of the LAMP primers for each of the selected turf grass pathogens using LAMP Designer 1.14 (PREMIER Biosoft). Therefore different parameters were tested to get different primer sets per organisms and loci (See TABLE 3 for a correlation of turf pathogen, selected loci and SEQ ID of best sequences used for primer design). The designed primers sets shown in TABLE 1 were then tested for their specificity (TABLE 3) and sensitivity (TABLE 4).

TABLE 1

| DNA SEQ ID | | |
|---|---|---|
| (Primer Sets) | Primer SEQ ID NO. | Primer name |
| 1 | 14 | B3 |
| | 15 | BIP |
| | 16 | F3 |
| | 17 | FIP |
| | 18 | LB |
| | 19 | LF |
| 2 | 20 | B3 |
| | 21 | BIP |
| | 22 | F3 |
| | 23 | FIP |
| | 24 | LB |
| | 25 | LF |
| 3 | 26 | B3 |
| | 27 | BIP |
| | 28 | F3 |
| | 29 | FIP |
| | 30 | LB |
| | 31 | LF |
| 4 | 32 | F3 |
| | 33 | B3 |
| | 34 | LF |
| | 35 | LB |
| | 36 | FIP |
| | 37 | BIP |
| 5 | 38 | F3 |
| | 39 | B3 |
| | 40 | LF |
| | 41 | LB |
| | 42 | FIP |
| | 43 | BIP |
| 6 | 44 | F3 |
| | 45 | B3 |
| | 46 | LF |
| | 47 | LB |
| | 48 | FIP |
| | 49 | BIP |
| 7 | 50 | F3 |
| | 51 | B3 |
| | 52 | LF |
| | 53 | LB |
| | 54 | FIP |
| | 55 | BIP |
| 8 | 56 | F3 |
| | 57 | B3 |
| | 58 | LF |
| | 59 | LB |
| | 60 | FIP |
| | 61 | BIP |
| 9 | 62 | F3 |
| | 63 | B3 |
| | 64 | LF |
| | 65 | LB |
| | 66 | FIP |
| | 67 | BIP |
| 10 | 68 | F3 |
| | 69 | B3 |
| | 70 | LF |
| | 71 | LB |
| | 72 | FIP |
| | 73 | BIP |
| 11 | 74 | F3 |
| | 75 | B3 |
| | 76 | LF |
| | 77 | LB |
| | 78 | FIP |
| | 79 | BIP |
| 12 | 80 | F3 |
| | 81 | B3 |
| | 82 | LF |
| | 83 | LB |
| | 84 | FIP |
| | 85 | BIP |
| 13 | 86 | F3 |
| | 87 | B3 |
| | 88 | LF |
| | 89 | LB |
| | 90 | FIP |
| | 91 | BIP |

### Specificity

To examine the specificity of the reaction (Literature see below), assays using the designed primer sets are tested using pure genomic DNA extracts from the fungal isolates described in TABLE 2. A comprehensive collection of different turf grass pathogens from distinct geographical origins were collected and grow on different media (potato dextrose/malt/cornmeal/cherry/V8). A ten-day old fungal culture was used to extract the DNA from mycelium (NucleoSpin Plant II—MACHEREY—NAGEL). The genomic DNA was diluted with nuclease free water to 5 ng/μl and a portion of 2.5 μl was used for the specificity tests.

The LAMP specificity tests were performed on a LightCycler 480 (Roche) in 96 well plates at 64° C. for 55 min. The amplicon-specific annealing temperature was determined during cooling from 98° C. to 65° C. with a ramp rate of −0.1° C. per second. Real-time LAMP assays were carried out in 10 μl reaction mixtures containing 5 μl of isothermal master mix at a 1× concentration (Optigene), 0.4 μM each external primer, 1.6 μM each internal primer, and 0.8 μM each loop primer (synthesized by Microsynth) and 2.5 μl of genomic DNA.

All reactions were carried out in duplicate and at two different days.

### LITERATURE FOR PERFORMING SPECIFICITY

Besuschio, S. A., Murcia, M. L., Benatar, A. F., Monnerat, S., Cruz, I., Picado, A., Schijman, A. G. (2017). Analytical sensitivity and specificity of a loop-mediated isothermal amplification (LAMP) kit prototype for detection of Trypanosoma cruzi DNA in human blood samples. PLOS Neglected Tropical Diseases, 11(7), e0005779.
Kitamura, M., Aragane, M., Nakamura, K., Watanabe, K., & Sasaki, Y. (2016). Development of Loop-Mediated Isothermal Amplification (LAMP) Assay for Rapid Detection of Cannabis sativa. Biological and Pharmaceutical Bulletin, 39(7), 1144-1149.
Seki, M., Kilgore, P. E., Kim, E. J., Ohnishi, M., Hayakawa, S., & Kim, D. W. (2018). Loop-Mediated Isothermal Amplification Methods for Diagnosis of Bacterial Meningitis. Frontiers in Pediatrics, 6.
Wang, D.-G., Brewster, J. D., Paul, M., & Tomasula, P. M. (2015). Two Methods for Increased Specificity and Sensitivity in Loop-Mediated Isothermal Amplification. Molecules, 20(4), 6048-6059.

TABLE 2

| ID | Microorganism | Strain number |
|---|---|---|
| 1 | Colletotrichum cereale | Stein 13-421 |
| 2 | Colletotrichum cereale | Stein UKCC1 |
| 3 | Colletotrichum cereale | Stein 13-394 |
| 4 | Colletotrichum cereale | Stein 13-396 |
| 5 | Colletotrichum cereale | Stein 13-415 |
| 6 | Colletotrichum cereale | Stein 871 |
| 7 | Colletotrichum graminicola | CBS 113173 |
| 8 | Colletotrichum graminicola | CBS 130836 |
| 9 | Gaeumannomyces graminis | Stein 870 |
| 10 | Gaeumannomyces graminis var. avenae | CBS 187.65 |
| 11 | Gaeumannomyces graminis var. avenae | Stein 880 |
| 12 | Gaeumannomyces graminis var. avenae | CBS 870.73 |
| 13 | Gaeumannomyces graminis var. graminis | CBS 387.81 |
| 14 | Gaeumannomyces graminis var. graminis | CBS 235.32 |
| 15 | Gaeumannomyces graminis var. graminis | CBS 903.73 |
| 16 | Gaeumannomyces graminis var. tritici | Stein 334 |
| 17 | Gaeumannomyces graminis var. tritici | CBS 186.65 |
| 18 | Gaeumannomyces graminis var. tritici | CBS 247.29 |
| 19 | Magnaporthe poae | CBS 131396 |
| 20 | Magnaporthe poae | CBS 131395 |
| 21 | Microdochium nivale majus | Stein 529 |
| 22 | Microdochium nivale nivale | Stein 72 |
| 23 | Microdochium nivale var. nivale | Stein 868 |
| 24 | Microdochium nivale var. nivale | Stein UKMN1 |
| 25 | Microdochium nivale var. nivale | Stein MN12055 |
| 26 | Pythium aphanidermatum | CBS 164.68 |
| 27 | Pythium aphanidermatum | Stein 889 |
| 28 | Pythium aphanidermatum | Stein K5902 |
| 29 | Pythium aphanidermatum | Stein 186 |
| 30 | Pythium aphanidermatum | Stein K6179 |
| 31 | Pythium aphanidermatum | Stein 620 |
| 32 | Pythium ultimum | CBS 122650 |
| 33 | Pythium ultimum var. sporangiiferum | CBS 219.65 |
| 34 | Pythium ultimum var. ultimum | CBS 305.35 |
| 35 | Pythium ultimum var. ultimum | Stein 71 |
| 36 | Pythium ultimum var. ultimum | Stein 146 |
| 37 | Pythium ultimum var. ultimum | CBS 378.34 |
| 38 | Pythium ultimum var. ultimum | CBS 725.94 |
| 39 | Pythium ultimum var. ultimum | CBS 726.94 |
| 40 | Pythium ultimum var. ultimum | Stein K6772 |
| 41 | Pythium ultimum var. ultimum | Stein K6773 |
| 42 | Rhizoctonia solani AG1.1C | CBS 109195 |
| 43 | Rhizoctonia solani AG2-2IV | CBS 109196 |
| 44 | Rhizoctonia solani AG4 | CBS 253.29 |
| 45 | Rhizoctonia solani AG | Stein 160 |
| 46 | Rhizoctonia solani AG2-2IIIB | Stein 722 |
| 47 | Rhizoctonia solani AG1-1A | Stein 184 |
| 48 | Rhizoctonia solani AG1-1 | ZHAW 103 |
| 49 | Rhizoctonia solani AG1-1A | CBS 101759 |
| 50 | Rhizoctonia solani AG1-1B | CBS 101761 |
| 51 | Rhizoctonia solani AG1-1C | CBS 101762 |
| 52 | Rhizoctonia solani AG1-1A | CBS 205.84 |
| 53 | Rhizoctonia solani AG1-1B | CBS 324.84 |
| 54 | Rhizoctonia solani AG2-2IIIB | CBS 101765 |
| 55 | Rhizoctonia solani AG4 | CBS 319.33 |
| 56 | Sclerotinia homoeocarpa | CBS 510.89 |
| 57 | Sclerotinia homoeocarpa | Stein 867 |
| 58 | Sclerotinia homoeocarpa | Stein 869 |
| 59 | Sclerotinia homoeocarpa | Stein UKSH1 |
| 60 | Sclerotinia homoeocarpa | Stein UKSH2 |
| 61 | Sclerotinia homoeocarpa | Stein UKSH3 |
| 62 | Sclerotinia homoeocarpa | Stein 13-392 |
| 63 | Sclerotinia homoeocarpa | Stein 13-410 |
| 64 | Sclerotinia homoeocarpa | Stein S-9 |
| 65 | Sclerotinia homoeocarpa | Stein S-83 |
| 66 | Thanatephorus cucumeris/Rhizoctonia solani | CBS 251.31 |
| | AG3 | |
| 67 | Thanatephorus cucumeris/Rhizoctonia solani | SYN 866 |
| | AG2-2IIIB | |
| 68 | Thanatephorus cucumeris/Rhizoctonia solani | Stein 184 |
| | AG1-1A | |
| 69 | Thanatephorus cucumeris/Rhizoctonia solani | Stein 689 |
| | AG4 | |

Stein and SYN strains: Syngenta, CH-4332 Stein, Switzerland
CBS strains: Westerdijk Fungal Biodiversity Institute, Utrecht, The Netherlands
ZHAW strains: Zurich University of Applied Sciences, Postfach 8820, Wädenswil, Switzerland

### Interpretation of the Results

As summarized in TABLE 3, the specificity of the LAMP assay was checked against the designed specific target for the fungi strains listed in TABLE 2. As an additional confirmation of specificity, a matching melting temperature of 82.6-89.9° C.±0.5° C. was observed for the different amplified products as also shown in TABLE 3.

TABLE 3

| | | | | Tm +/− 0.5° C. | Positive reaction |
|---|---|---|---|---|---|
| | | | Primer set | (melting | with organisms |
| Turf Grass Pathogen | DNA Loci | DNA of SEQ ID NO. | (Table 1) | temperature) | (Table 2) |
| Sclerotinia homoeocarpa | Elongation factor 1-alpha | 1 | 1 | 87.1 | 56-65 |
| Rhizoctonia solani AG2-2IIIB | Internal Transcribed Spacer | 2 | 2 | 86.0 | 43, 46, 48, 54, 55, 67 |
| Rhizoctonia solani AG2-2IV | Beta-Tubulin | 9 | 9 | 89.9 | 43 |
| Microdochium nivale var. | Beta-Tubulin | 3 | 3 | 88.9 | 22-25 |
| nivale | | | | | |
| Pythium aphanidermatum | Beta-Tubulin | 4 | 4 | 89.3 | 26-31 |
| Pythium aphanidermatum | Cytochrome c oxidase subunit 1 | 10 | 10 | 82.6 | 26-31, 32-33 |
| Gaeumannomyces graminis | Beta-Tubulin | 5 | 5 | 89.7 | 10-18 |
| var. Avenae | | | | | |
| Gaeumannomyces graminis | Beta-Tubulin | 8 | 8 | 88.2 | 10-18 |
| var. Avenae | | | | | |
| Microdochium nivale var. | Beta-Tubulin | 6 | 6 | 89.2 | 21 |
| majus | | | | | |
| Magnaporthe poae | Beta-Tubulin | 7 | 7 | 88.4 | 19-20 |
| Colletotrichum graminicola | Superoxide Dismutase | 11 | 11 | 88.9 | 7-8 |
| Colletotrichum cereale | Superoxide Dismutase | 12 | 12 | 89.6 | 2-6 |
| Pythium ultimum var. ultimum | Large Subunit Nuclear Ribosomal | 13 | 13 | 88.8 | 33-41 |
| | RNA | | | | |

The sensitivity of the of the described primer sets corresponding to the DNA of Seq ID Nos. 1-13 (Table 1) were determined using serial dilutions of genomic DNA (1 ng to 100 fg) of all fungi of interests, with each reaction made in duplicate at two different days. Pure genomic DNA from all fungi was obtained using the NucleoSpin Plant II (MACHEREY-NAGEL). The LAMP sensitivity tests were performed on a LightCycler 480 (Roche) in 96 well plates at 6400 for 55 min. The amplicon-specific annealing temperature was determined during cooling from 9800 to 6500 with a ramp rate of −0.1° C. per second. Real-time LAMP assays were carried out in 10 μl reaction mixtures containing 5 μl of isothermal master mix at a 1× concentration (Optigene), 0.4 μM each external primer, 1.6 μM each internal primer, and 0.8 μM each loop primer (synthesized by Microsynth) and 2.5 μl of genomic DNA.

TABLE 4

| | Tm +/− 0.5° C. | Sensitivity/detection limit of |
|---|---|---|
| DNA of SEQ ID NO. | (melting temperature) | genomic DNA |
| 1 | 87.1 | 2.5 picogram |
| 2 | 86.0 | 2.5 picogram |
| 3 | 88.9 | 250 picogram |
| 4 | 89.3 | 25 picogram |
| 5 | 89.7 | 25 picogram |
| 6 | 89.2 | 25 picogram |
| 7 | 88.4 | 25 picogram |
| 8 | 88.2 | 2.5 picogram |
| 9 | 89.9 | 250 picogram |
| 10 | 82.6 | 25 picogram |
| 11 | 88.9 | 25 picogram |
| 12 | 89.6 | 25 picogram |
| 13 | 88.8 | 2.5 picogram |

### Method of Detecting Fungal Pathogen in a Turf Grass Sample

### Sample Collection

A tuft of turf sample including grass roots is collected at a location where a fungal pathogen is expected. The turfgrass may also show symptoms. The turf sample is placed in a clean 50 ml tube (Corning) and stored at −20° C. until use. DNA is extracted using Plant Material Lysis Kit (Optigene). A 1 cm³ cube of the turf sample is placed into a Bijou tube containing 1 ml of lysis buffer (Optigene). The homogenization of the turf sample is conducted by shaking the Bijou tube for 1 min. A volume of 10 μl of the lysate is transferred into a dilution tube provided (Optigene) and mixed vigorously by shaking. The diluted lysate is subsequently defined as the template.

### LAMP Reaction

In some embodiments, the LAMP reaction is performed at about 60° C. to about 70° C., such as about 64° C. to about 67° C., or about 64° C. to about 66° C. In specific examples, the LAMP reaction is performed at 64° C.

In some embodiments, the LAMP reaction is allowed to proceed for about 15 to about 45 minutes, such as about 20 minutes to about 40 minutes, or about 25 minutes to about 35 minutes.

In some embodiments, the concentration of primers in the LAMP reaction according to the present invention is 1.4-1.8 μM, more specifically 1.6 μM for the forward (FIP) and reverse (BIP) inner primers, 0.2-0.4 μM, more specifically 0.4 μM for forward (F3) and reverse (B3) outer primers, and 0.4-0.8 μM, more specifically 0.8 μM, loop forward (LF) and/or loop back (LB) primers that are useful to accelerate amplification of nucleic acid present in the turf sample and to reduce the detection time of any target fungi DNA that may be present in such turf sample.

### Suitable Buffer Systems Useful in the Reaction of LAMP Assay Include:

1× Isothermal Amplification Buffer Pack from New England Biolabs
20 mM Tris-HCl
10 mM (NH₄)₂SO₄
50 mM KCl
2 mM MgSO₄
0.1% Tween@20
(pH 8.8 @25° C.)
1× Isothermal Amplification Buffer II Pack from New England Biolabs
20 mM Tris-HCl
10 mM (NH₄)₂SO₄
150 mM KCl
2 mM MgSO₄
0.1% Tween@20
(pH 8.8 @25° C.)

### Suitable Enzyme Systems (DNA Polymerase, Etc.) Useful in the Reaction of LAMP Assay Include:

| Distributor | catalog | Product name |
|---|---|---|
| New England | M0374 | Bst 3.0 DNA Polymerase |
| Biolabs | | |
| New England | M0537 | Bst 2.0 DNA Polymerase |
| Biolabs | | |
| New England | M0538 | Bst 2.0 WarmStart ® DNA Polymerase |
| Biolabs | | |
| New England | M0275 | Bst DNA Polymerase, Large Fragment |
| Biolabs | | |
| Lucigen | 30066 | LavaLAMP ™ DNA Master Mix |
| Lucigen | 30067 | LavaLAMP ™ DNA Master Mix with Dye |
| Eiken | LMP204 | DNA Amplification Kit |
| Eiken | LMP207 | Dried DNA Amplification Reagent |
| Optigene | ISO-001 | FAST isothermal amplification with dye |
| Optigene | ISO-001nd | FAST isothermal amplification |
| Optigene | ISO-DR001 | FAST isothermal amplification with dye, dried |
| Optigene | ISO-004 | FASTEST isothermal amplification with dye |
| Optigene | ISO-004nd | FASTEST isothermal amplification |
| Optigene | ISO-DR004 | FASTEST isothermal amplification with dye, dried |
| Optigene | ISO-001Tin | HIGHLY THERMOSTABLE enzyme suitable for isothermal |
| | | amplification with dye |
| Optigene | ISO- | HIGHLY THERMOSTABLE enzyme suitable for isothermal |
| | DR001Tin | amplification with dye, dried |

In one embodiment, the LAMP reactions are performed on a Genie instrument (Optigene) in a test strip with dried reagents (Optigene). In one embodiment, the strips have eight 150 μl wells (2 control and 6 for assays). Real-time LAMP assays are carried out in 25 μl reaction mixtures containing 15 μl of isothermal master mix at a 1× concentration (Optigene), 0.4 μM each external primer, 1.6 μM each internal primer, and 0.8 μM each loop primer (synthesized by Microsynth) selected from at least one of the primer sets of Table 1. Prior to adding the template, the lyophilized reaction strip is resuspended in 22 μl resuspension buffer (Optigene). All test strips include a negative control and a positive plant control primer set provided by Optigene. For all assays, 3 μl of template is added per reaction and well. The reaction is held at 64° C. for 30-55 min followed by an anneal program. The temperature profile of the anneal program is determined during cooling from 98° C. to 65° C. with a ramp rate of −0.1° C. per second.

The isothermal master mix contains a fluorescent double-stranded DNA binding dye to permit the real-time detection of the amplicons. The assays are optimized in terms of reaction time, temperature, and the volume of DNA added per reaction.

The fluorescence data that is acquired during amplification phase at 64° C. is reported as amplification time. The fluorescence derivative data that is acquired during the anneal phase is reported as an annealing temperature.

Alternatively, the LAMP assay reaction does not include an anneal program in which case a pH-sensitive indicator dye can be used to assess the presence of target fungal DNA. In some examples, the pH-sensitive indicator dye is a colored dye detectable in visible light. In particular examples, the colored dye comprises cresol red, phenol red, m-cresol purple, bromocresol purple, neutral red, naphtholphthalein, thymol blue or naphtolphthalein. In other examples, the pH-sensitive indicator dye is a fluorescent indicator dye. In particular examples, the fluorescent dye comprises 2′,7′-bis-(2-carboxyethyl)-5(6)-carboxyfluorescein, 5(6)-carboxy-2′,7′-dichlorofluorescein, 5(6)-carboxyfluorescein, 3,6-diacetoxyphthalonitrile, 6,8-dihydroxy-1,3-pyrenedisulfonic acid, or 5-(and-6)-carboxyl seminaphthorhodafluor.

Following the foregoing procedures, the detection of the presence of fungal pathogen DNA (Table 3) in a turf sample may indicate the presence of a turf fungal pathogen that may cause relevant turf diseases (including, for example, anthracnose, take-all patch, summer patch, snow mold, pythium blight, brown patch and dollar spot). Early and efficient detection provides suitable turf grass disease management decisions to be undertaken.

1. A method for detecting fungal DNA in a turf grass sample with a loop-mediated isothermal amplification (LAMP) assay which contains primers for fungal DNA of at least one turf pathogenic fungi selected from Pythium aphanidermatum, Gaeumannomyces graminis spp., Microdochium nivale spp., Magnaporthe poae, Colletotrichum graminicola, Colletotrichum cereale and Pythium ultimum var. ultimum, comprising:
subjecting the turf sample to a LAMP reaction wherein the LAMP reaction uses a primer set of four or more nucleic acid sequences with each primer in the set having from 15 to 50 nucleic acids, and wherein the set of primers comprises at least one primer set selected from:
(c) a primer set for detecting Microdochium nivale var. nivale DNA that comprises or is selected from primers each respectively having a sequence which is at least 90% identical to SEQ ID NO: 26, 27, 28, and 29.;
(d) a primer set for detecting Pythium aphanidermatum DNA that comprises or is selected from primers each respectively having a sequence which is at least 90% identical to SEQ ID NO: 32, 33, 36, and 37.;
(e) a primer set for detecting Gaeumannomyces graminis var. avenae DNA that comprises or is selected from primers each respectively having a sequence which is at least 90% identical to SEQ ID NO: 38, 39, 42 and 43
(f) a primer set for detecting Microdochium nivale var. majus DNA that comprises or is selected from primers each respectively having a sequence which is at least 90% identical to SEQ ID NO: 44, 45, 48 and 49;
(g) a primer set for detecting Magnaporthe poae DNA that comprises or is selected from primers each respectively having a sequence which is at least 90% identical to SEQ ID NO: 50, 51, 54 and 55;
(h) a primer set for detecting Colletotrichum graminicola DNA that comprises or is selected from primers each respectively having a sequence which is at least 90% identical to SEQ ID NO: 74 75, 78 and 79;
(i) a primer set for detecting Colletotrichum cereale DNA that comprises or is selected from primers each respectively having a sequence which is at least 90% identical to SEQ ID NO: 80, 81, 84 and 85; and
(j) a primer set for detecting Pythium ultimum var. Ultimum DNA that comprises or is selected from primers each respectively having a sequence which is at least 90% identical to SEQ ID NO: 86, 87, 90 and 91.

2.-4. (canceled)

5. A method according to claim 1 wherein, the primer set for detecting Microdochium nivale var. nivale DNA comprises or is selected from primers each respectively having a sequence which is at least 90% identical to SEQ ID Nos: 26, 27, 28, 29, 30 and 31.

6. A method according to claim 1 wherein, the primer set for detecting Pythium aphanidermatum DNA comprises or is selected from primers each respectively having a sequence which is at least 90% identical to SEQ ID NOs: 32, 33, 34, 35, 36 and 37.

7. A method according to claim 1 wherein, the primer set for detecting Pythium aphanidermatum DNA comprises or is selected from primers each respectively having a sequence which is at least 90% identical to SEQ ID NOs: 68, 69, 70, 71, 72 and 73.

8. A method according to claim 1 wherein, the primer set for detecting Gaeumannomyces graminis var. avenae DNA comprises or is selected from primers each respectively having a sequence which is at least 90% identical to SEQ ID NOs: 38, 39, 40, 41, 42 and 43.

9. A method according to claim 1 wherein, the primer set for detecting Gaeumannomyces graminis var. avenae DNA comprises or is selected from primers each respectively having a sequence which is at least 90% identical to SEQ ID NO: 56, 57, 58, 59, 60 and 61.

10. A method according to claim 1 wherein, the primer set for detecting Microdochium nivale var. majus DNA comprises or is selected from primers each respectively having a sequence which is at least 90% identical to SEQ ID Nos: 44, 45, 46, 47, 48 and 49.

11. A method according to claim 1 wherein, the primer set for detecting Magnaporthe poae DNA comprises or is selected from primers each respectively having a sequence which is at least 90% identical to SEQ ID NOs: 50, 51, 52, 53, 54, and 55.

12. A method according to claim 1 wherein, the primer set for detecting Colletotrichum graminicola DNA comprises or is selected from primers each respectively having a sequence which is at least 90% identical to SEQ ID NOs:74, 75, 76, 77, 78 and 79.

13. A method according to claim 1 wherein, the primer set for detecting Colletotrichum cereale DNA comprises or is selected from primers each respectively having a sequence which is at least 90% identical to SEQ ID Nos 80, 81, 82, 83, 84 and 85.

14. A method according to claim 1 wherein, the primer set for detecting Pythium ultimum var. Ultimum DNA comprises or is selected from primers each respectively having a sequence which is at least 90% identical to SEQ ID Nos: 86, 87, 88, 89, 90 and 91.

15. A kit for the detection of fungal DNA in a turf grass sample using a LAMP assay, comprising one or more than one of the primer sets identified in claim 1.

16. The kit of claim 15, further comprising buffer, DNA polymerase.
